(19) United States
(12) Patent Application Publication
(10) Pub. No.: US 2024/0184137 A1
(43) Pub. Date: Jun. 6, 2024

(54) CONTACT LENSES AND METHODS OF MAKING CONTACT LENSES

(71) Applicant: University of Florida Research Foundation, Inc., Gainesville, FL (US)

(72) Inventors: Anuj CHAUHAN, Gainesville, FL (US); Poorvajan SEKAR, Gainesville, FL (US)

(21) Appl. No.: 18/285,518

(22) PCT Filed: Apr. 12, 2022
(86) PCT No.: PCT/US2022/024388

§ 371 Date: Oct. 4, 2023

Related U.S. Application Data
(60) Provisional application No. 63/174,814, filed on Apr. 14, 2021.

(51) Int. Cl. G02C 7/04; A61F 2/16; G02B 1/04

(52) U.S. Cl. CPC G02C 7/04; A61F 2/16; G02B 1/043

(57) ABSTRACT

Aspects of the present disclosure provides for contact lenses that employing Vitamin-E integrated contact lenses as a vehicle for treating conditions (e.g., bacterial keratitis and post-surgical inflammation) through at least one medicament delivery (e.g., a triumvirate medicament delivery such as a triplicate of antibiotic and anti-inflammatory drugs) to the ocular surface.

### CROSS-REFERENCE TO RELATED APPLICATIONS

This application claims priority to, co-pending U.S. Patent Application entitled “CONTACT LENSES AND METHODS OF MAKING CONTACT LENSES,” filed on Apr. 14, 2021, and assigned application No. 63/174,814, each of which are incorporated herein by reference in their entireties.

### BACKGROUND

Corneal damage is also induced by surgical treatment procedures including laser in situ keratomileusis (LASIK), which involves potential flap dislocation during refractive error correction. PRK surgery, like its former surgical counterpart, involves modification of lens curvature by excimer laser treatment. Post-FDA approval in 1995, 1 million procedures are performed annually in the US alone. For postoperative care, patients are generally prescribed a course of antibiotic and anti-inflammatory drugs, usually administered to the operated corneal surface in the form of topical eye-drops. Topical drop application is continued as protocol till re-epithelialization of the corneal surface reaches maturity. In addition to topical treatment, patients are advised to wear protective devices including tinted eyeglasses to minimize light-induced epithelial cell disruption. In addition to screening peripheral glares from external surroundings or an illuminated indoor setting, protective googles prevents entry to ambient dust particulates that induce voluntary rubbing of the eyelid. These surgical surface ablation procedures involve making an incision of the outer corneal tissue, dislodged from the surface as a corneal flap to treat refractive errors. Post-operative treatment procedures rely on synthetic scaffolds to secure the loosely adherent flap to promote re-epithelization of surface. Bandage contact lenses are one such scaffolding materials used in refractive surgery to reduce inflammatory cell infiltration, reduce corneal scarring, basement membrane reconstruction, and restore epithelial stromal adhesion. Though BCLs secure the flap's position and promote re-epithelialization, frequent re-insertion of these lenses after topical drop administration can induce flap misalignment and tenting called striae folds. Striae folds, prevalent among 3-5% of the post-operated elderly population can cause result is decreased visual acuity along with pain and discomfort induced by frequent blinking.

### SUMMARY

Embodiments of the present disclosure provides for contact lenses that employ Vitamin-E integrated contact lenses as a vehicle for treating conditions (e.g., bacterial keratitis and post-surgical inflammation) through at least one medicament delivery (e.g., a triumvirate medicament delivery such as a triplicate of antibiotic and anti-inflammatory drugs) to the ocular surface.

The present disclosure provides for a contact lens, comprising: a silicone-hydrogel-based lens comprising Vitamin E and at least one medicament, wherein the medicament includes an antibiotic medicament, a steroidal anti-inflammatory medicament, and a non-steroidal anti-inflammatory medicament, or combination thereof, wherein contact lens is characterized as having a sustained release of the at least one medicament.

The present disclosure provides for a contact lens, comprising: a hydrogel-based lens comprising Vitamin E and ketoralac and at least one medicament, wherein the medicament includes an antibiotic medicament, a steroidal anti-inflammatory medicament, and a non-steroidal anti-inflammatory medicament, or combination thereof, wherein contact lens is characterized as having a sustained release of the at least one medicament.

The present disclosure provides for a method of preparing a hydrogel-based contact lens, the method comprising: exposing the hydrogel-based contact lens to a first solution including Vitamin E and a solvent, wherein the solvent swells a hydrogel of the hydrogel-based contact lens so that the Vitamin becomes entrapped by the hydrogel to form a modified contact lens; exposing the modified contact lens to a second solution including at least one medicament absorbed into the lens matrix to form a final contact lens, wherein the medicament includes an antibiotic medicament, a steroidal anti-inflammatory medicament, and a non-steroidal anti-inflammatory medicament, or combination thereof, wherein the final contact lens is characterized as having a sustained release of the at least one medicament.

The present disclosure provides for a method of treating an ocular condition, comprising: positioning a contact lens onto the eye in need of treatment for a period of time, wherein the contact lens is a contact lens as described in any one of claims 1 to 10; and removing the contact lens after the period of time has expired.

Other devices, methods, features, and advantages will be or become apparent to one with skill in the art upon examination of the following drawings and detailed description. It is intended that all such additional devices, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates transmittance spectra of an 80 μm thick commercial ACUVUE® OASYS® control lens and the modified version after vitamin-E (α-tocopherol) incorporation. Integration of vitamin-E into the lens matrix does not have an impact on transparency of the commercial lenses.

FIG. 2A-2F illustrae the dynamics of Levofloxacin, Ketoralac, and Dexamethasone transport during the release phase in commercial ACUVUE® OASYS® a,c,e). The corresponding mass percentages are depicted in figures b), d), and f) respectively. “Release percentage” shown for bimatoprost transport is calculated as the ratio of cumulative drug uptake at any time and that after equilibrium is achieved. Data is presented as mean±SD with n=3.

FIGS. 3A-3B illustrate Vitamin E nanobarriers at 30% prolong the release duration of levofloxacin (FIG. 3A), dexamethasone (FIG. 3B), and ketorolac compared (FIG. 3C) to the control. The release of each of the drug loaded separately (IR: individual release) is comparable to the release of the same drug when loaded with the other two drugs (CR). Drug was loaded in lenses by soaking in 80 μg dexamethasone, 5 mg/mL ketorolac and 25 mg/mL levofloxacin. Data is mean±std (n=3). Solid lines represent fits to the data based on diffusion control release. The model fits the data well with best fit diffusivity values and partition coefficients included in the legends.

FIG. 4A-4C illustrate predicted aqueous humor concentrations for contact lenses and eye drops. The MIC and EC values are included for comparison. Also predicted concentrations for commercial dexamethasone releasing devices are included.

FIGS. 5A-5B illustrate in vitro drug release from control SENOFILCON A (FIG. 5A) and 20% VE loaded SENOFILCON A (FIG. 5B) lenses under sink conditions. Drug was loaded by soaking the lenses in 5 mg/mL (top) and 20 mg/mL (bottom) of moxifloxacin dissolved in phosphate buffered saline (1×). Drug release was measured by soaking the drug loaded lenses in 3 mL of phosphate buffered saline (1×).

FIG. 6 illustrates a graph regarding the delivery of bromfenac by loading the drug in Acuvuce Oasys with 20% vitamin E lenses in a 3 mL solution of bromfenac at 0.5, 1, 2 and 10 mg/mL.

### DETAILED DESCRIPTION

The present disclosure provides for contact lenses that employing Vitamin-E integrated contact lenses as a vehicle for treating conditions (e.g., bacterial keratitis and post-surgical inflammation) through at least one medicament delivery (e.g., a triumvirate medicament delivery such as a triplicate of antibiotic and anti-inflammatory drugs) to the ocular surface. The contact lenses of the present disclosure including the medicament(s) do not significantly alter their individual diffusive properties and there is minimal interaction when multiple medicaments are present at a physiological pH of 7.4. The high specific surface area of the bi-continuous matrices and high aspect ratios of the phase separated Vitamin-E barriers play a crucial role in sustained release of the drug(s).

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”. In regard to use of “consisting essentially of” in regard to colorants, components that do not effect or substantially effect the color to be imparted to the hydrogel or the silicone-hydrogel and/or contact lenses or can be present in an amount that is considered an impurity for the preparation of hydrogels or the silicone-hydrogel or contact lenses.

As used in the specification and the appended claims, the singular forms “a,” “an” and “the” include plural referents unless the context clearly dictates otherwise. Thus, for example, reference to “a colorant,” or “a lens,” including, but not limited to, two or more such pigments, colorants, or lenses, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g. about 1%, about 2%, about 3%, and about 4%) and the sub-ranges (e.g. about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a colorant refers to an amount that is sufficient to achieve the desired improvement in the property modulated by the formulation component, e.g. achieving the desired level of blocking of a desired wavelength. The specific level in terms of wt% in a composition required as an effective amount will depend upon a variety of factors including the amount and type of colorant, type of hydrogel or the silicone-hydrogel, level of target blocking of a desired wavelength, and end use of the hydrogel or the silicone-hydrogel made using the composition.

As used herein, the terms “optional” or “optionally” means that the subsequently described event or circumstance can or cannot occur, and that the description includes instances where said event or circumstance occurs and instances where it does not.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as glaucoma, inflammation, ophthalmological bacterial or fungal infections, macular edema, and/or diabetic retinopathy. The effect can be therapeutic in terms of a partial or complete cure of a disease, condition, symptom or adverse effect attributed to the disease, disorder, or condition. The term “treatment” as used herein can include any treatment of glaucoma, inflammation, ophthalmological bacterial or fungal infections, macular edema, and/or diabetic retinopathy in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1-to-C6 alkyl esters and C5-to-C7 cycloalkyl esters, although C1-to-C4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C1-to-C6 alkyl amines and secondary C1-to-C6 dialkyl amines. In the case of secondary amines, the amine can also be in the form of a 5- or 6-membered heterocycle containing one nitrogen atom. Amides derived from ammonia, C1-to-C3 alkyl primary amides and C1-to-C2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Unless otherwise specified, temperatures referred to herein are based on atmospheric pressure (i.e. one atmosphere).

### Discussion

Embodiments of the present disclosure provide for contact lenses including Vitamin E and one or more medicaments, methods of making contact lenses, methods of treating ocular conditions, and the like. Embodiments of the present disclosure can be used to administer one or more medicaments in a sustained release (e.g., over 3 to 7 days depending upon the application and intended goals) manner using Vitamin E, where when multiple medicaments are present, the medicaments do not interfere with one another. In particular, embodiments of the present disclosure can be used to treat ocular conditions (e.g., disease, post-surgery application to improve recovery and reduce the cause of problems such as infection) over a period of time (e.g., 3 to 7 days) by providing the medicament(s) in a sustained release manner using Vitamin E.

In an aspect, the present disclosure provides for a contact lens (e.g., a hydrogel or silicone-hydrogel-based lens) that includes entrapped Vitamin E within the hydrogel and at least one medicament on the surface and/or within hydrogel. In a particular example the medicament is a non-steroidal anti-inflammatory medicament such as ketorolac. Other medicaments include an antibiotic medicament, a steroidal anti-inflammatory medicament, and other non-steroidal anti-inflammatory medicaments. In an aspect, Vitamin E and three medicaments are entrapped in the hydrogel of the contact lens: an antibiotic medicament, a steroidal anti-inflammatory medicament, and a non-steroidal anti-inflammatory medicament. The inclusion of the Vitamin E gives the contact lens the characteristic of having a sustained release of the medicament(s) present in the coating of the contact lens.

The sustained release characteristic can be described as releasing a medicament(s) over a longer period of time relative to the same contact lens with the same amount of medicament(s) present but without the Vitamin E present. The amount of time of release can be 50%, 100%, 200%, 500%, 1000%, 10,000% or more than a contact lens without the Vitamin E. The sustained release can be for about 2 to 7 days or 3 to 5 days, or 3 to 4 days, where the medicaments present are released at a desirable level to achieve the desired function of the medicament (e.g., function as an anti-inflammatory). The release profiles for each medicament may be the same or different depending upon the design. For example, one medicament may be present at higher levels than another and release for a longer period of time and/or at higher levels. Depending upon the application and the subject (e.g., humans), the amount of Vitamin E present and/or the amount of each medicament present can be modified for the intended purpose of administering the medicament for the eye in need of treatment.

The contact lens can include those known in the art such as those made of a swellable hydrogel or a silicone-hydrogel. In an aspect the hydrogel based lens can be made from one or more of the following: hydroxyethyl methacrylate (HEMA), dimethyl acrylate (DMA), methacrylic acid, or 3-[tris (trimethylsiloxy)silyl]propyl methacrylate (Tris methacrylate). In an example the contact lens is a commercially available ACUVUE® OASYS® lenses (Johnson and Johnson Vision Care).

Vitamin E can include one or a combination of four tocopherols and/or four tocotrienols (e.g., alpha-, beta-, gamma-, and delta-tocopherol and alpha-, beta-, gamma-, and delta-tocotrienol). In an aspect, the Vitamin E can be a tocopherol stereoisomer such as DL-alpha tocopherol. The amount of Vitamin E present in a single contact lens can be about 1% to 70% or about 10% to 30% of the dry weight of the contact lens, where the amount present can be varied depending upon the patient, the eye condition in need of treatment, the type and/or amount of medicament present, the number of medicaments, and the like.

The medicament can include an antibiotic medicament (e.g., levofloxacin, moxifloxacin, chloramphenicol, ciprofloxacin, ofloxacin, gatifloxacin, besifloxacin, norfloxacin, bacitracin, tetracycline, neomycin, erythromycin, tobramycin, gentamicin, bacitracin, erythromycin, azithromycin, polymyxin B, trimethoprim), a non-steroidal anti-inflammatory medicament (e.g., ketorolac, bromfenac, diclofenac, nepafenac, flubiprofen), and steroidal anti-inflammatory medicament (e.g., dexamethasone, fluorometholone, fluocinolone, lotoprednol, difluprednate, triamcinolone, prednisolone). In an aspect, multiple different types of the antibiotic medicament, the non-steroidal anti-inflammatory medicament, and/or the steroidal anti-inflammatory medicament can be used. For example, two non-steroidal anti-inflammatory medicaments can be used with or without one or more of each of the antibiotic medicament and steroidal anti-inflammatory medicament. In an aspect the non-steroidal anti-inflammatory medicament is ketorolac, the antibiotic medicament is levofloxacin and steroidal anti-inflammatory medicament is dexamethasone. In an aspect, the medicaments are either one or multiple antibiotic drugs to treat ocular infections.

In an aspect, the present disclosure provides for methods of preparing a contact lens. The method can include exposing the contact lens to Vitamin E within a first solution for a period of time (e.g., about 1 min to 24 hours). The contact lens is soaked in the Vitamin E that is within a solution such as an organic liquid (e.g., ethanol, methanol, isopropyl alcohol, dichloromethane, chloroform, ethyl acetate, diethyl ether, and combinations thereof) for a few minutes to few days at about room temperature. The modified contact lens is then processed to remove the excess Vitamin E and alcohol and to reduce the contact lens to substantially the same size prior to treatment.

Vitamin E (α-tocopherol) nanobarriers are introduced by phase separation of vitamin E on the internal interface between the silicone and hydrogel phases in the silicone hydrogel lenses such as ACUVUE OASYS. The lens is supersaturated with vitamin E by soaking in vitamin E-ethanol solution followed by soaking in phosphate buffered saline. The loading of vitamin E in lens (f_{VE,l}) is five times that in the ethanol solution (f_{VE,e}), so soaking ACUVUE OASYS lenses in 4% vitamin E-ethanol solution results in a loading of 20% (w/w) on dry basis. The vitamin E barriers are disk-like with an average thickness and radius of 21 and 350 nm, respectively¹. The NB-CL are autoclaved for sterilization. The barriers are stable for >1 year due to the negligible solubility of vitamin E in aqueous solutions.

Next the modified contact lens is exposed to at least one medicament (e.g., such as those provided herein) in an amount to achieve the desired outcome to form a final contact lens. The modified contact lens is soaked for 1 hours to 10 days at about room temperature with a second solution of at least one medicament, where the contact lens absorbs and/or adsorbs the medicament(s). In an aspect, the at least one medicament can be within the contact lens (e.g., vitamin E) and/or on the surface of the contact lens. The final contact lens is characterized as having a sustained release of the at least one medicament. Additional details for making the contact lens are provided in Example 1.

In an aspect, the present disclosure provides for a method of treating an ocular condition (e.g., corneal keratitis, inflammation, or the like). The method includes positioning a contact lens as described herein onto the eye in need of treatment for a period of time (e.g., 2 to 7 days) to achieve the desired goal of treating or relieving the condition. If needed, the contact lens can be replaced with another contact lens with the same or different amounts of medicaments and/or the same or different types of medicaments. After the appropriate amount of time (e.g., the medicaments are used for example) the contact lens can be removed. If needed, additional supplemental treatment of the eye can be conducted (e.g., oral administration of medicament(s)). In an aspect, the contact lens can be used in a post procedure or operative setting where the eye is of need of treatment or prophylactic treatment.

In another aspect, the contact lens and the Vitamin E and/or medicament(s) are packaged individually and combined by the physician just prior to the therapy. In an aspect, the contact lens can be pre-treated with Vitamin E so the kit includes the medicaments. Additional components present can include one or more buffer components and a hydrating solution as well as instructions for use.

Having described the present disclosure in general, additional details are provided.

Eye drops are not optimal for treating many ophthalmic diseases. The present disclosure provides for delivering drugs by contact lenses to improve therapy for multiple diseases including post cataract management and infections.

Cataract surgery has become a very common procedure with about 10 million cataract operations each year in the world² with a significant increase expected due to aging of the population. The surgery could result in significant inflammation which is left untreated could cause complications such as cystoid macular oedema, increased intraocular pressure, posterior capsular opacification, chronic uveitis, fibrin formation³. Post-surgical management requires instillation of eye drops to deliver antibiotics, steroids and NSAID but this approach has many problems.

Eye drops have low ocular bioavailability. Eyedrops account for nearly 90% of marketed products for treating anterior segment ocular diseases; yet, eyedrops have poor patient compliance⁴⁻¹⁰ and low bioavailability¹¹. After instillation, an eye drop is rapidly cleared from the surface of the eye due to drainage of tears into the nasal sac. Within about 5 minutes of instillation the concentration on the eye surface decreases to negligible values. During this short time only a very small amount of drug (<5% of drug in the drops) diffuses into the cornea while the remaining almost 95% is lost to diffusion across the conjunctiva and drainage into the nasal sac¹¹. The drug that reaches into the cornea diffuses further into the anterior chamber from where it is cleared via aqueous outflow¹². The rapid precorneal clearance coupled with the aqueous humor clearance results in a rapid decrease in the drug concentration in the aqueous humor. The concentration reaches a maximum value soon after the instillation and then decreases to negligible values in a few hours, thereby necessitating multiple eyedrops in a day to maintain therapeutic concentrations.

Instillation of eye drops is difficult resulting in a potential for poor compliance¹³. The regimen of eye drops for post cataract patients includes steroids about 3-4× daily for 4-weeks, antibiotics about 4× daily for 2-weeks and NSAIDs 1× daily for 3-4 weeks. In a study with 30 patients after cataract (n=24) and glaucoma filtration surgery (n=6), the mean dose compliance was 50.2%, with below 25% compliance in approximately one out of five patients.¹⁴ In fact, in almost 93% cases, patents use incorrect technique to administer the eyedrops¹⁵. The lack of proper administration could cause additional problems such as corneal and conjunctival abrasions (68% of elderly study patients)¹⁶ and potential for wound gape and efflux of fluid into the eye following pressure on the eye¹⁷, which may result in an increased the risk of endophthalmitis from the eye drop treatment¹⁸.

These deficiencies have driven research in developing intracameral devices that can be placed in the anterior chamber after the surgery for sustained release of dexamethasone. The FDA has approved DEXYCU™ which is an emulsion formulation that provides sustained release of dexamethasone for 21 days^{19,20}. Clinical studies show safety, efficacy and significant patient acceptance with 68.7% in the treatment group agreeing that not using eyedrops was very convenient²¹. DEXTENZA® is a dexamethasone releasing puncta plug that is another commercialized device for delivery of the drug after cataract surgery¹⁸. While DEXYCU® and DEXTENZA® are improvements over eye drops, neither deliver all drugs and so patients may still require instillation of antibiotic eye drops to minimize the possibility of infections.

In an aspect, the present disclosure can address one or more of these problems by designing contact lenses that can deliver all three drugs at controlled rates for extended durations with high bioavailability making this significantly superior to the drop-based therapy as well as other commercial devices. It should be noted that while bandage contact lenses are not used commonly after cataract surgery, there are no contraindications and in fact multiple studies have suggested that bandage contact lenses can be safely used after cataract surgery²²⁻²⁴.

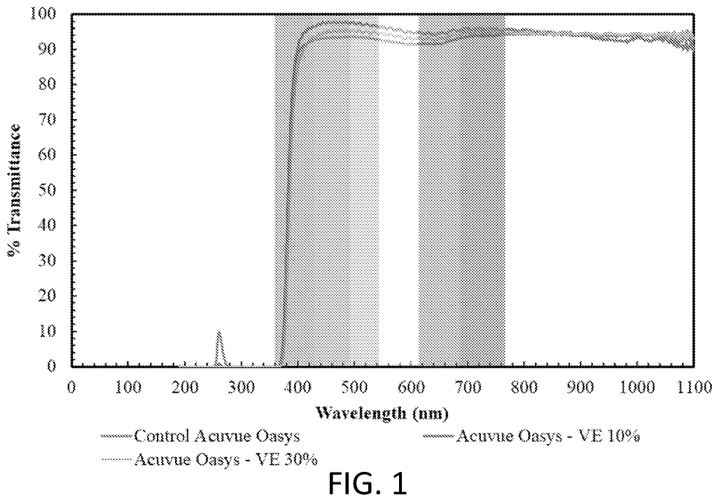

Contact lens-based delivery can offer advantages compared to the drop-based approach as provided below.
1. Contact lenses have about 50% bioavailability for drug delivered to the aqueous humor¹² compared to 1-5% for eye drops²⁵⁻²⁹ due to their location on the eye (FIG. 1) which results in a significant increase in residence time to about 30 min compared to a few minutes for drops.
2. Contact lens-based approach will reduce the need for patients to instill drugs which will improve compliance. The lenses will be inserted by the doctor which will also eliminate the need for the patients to be able to insert the lenses.
3. The more uniform concentration of the drugs will improve therapy.
4. Contact lenses will be preservative-free which will reduce patient exposure to preservatives which can cause toxicity.

### Other Applications of Aspects of the Present Disclosure

Photo refractive keratectomy (PRK) involves removal of the corneal epithelium and reshaping the anterior stroma. PRK patients are fitted with a bandage contact lens (BCL) to protect the abraded cornea from the shear stress exerted by the blink and/or rubbing of the eyes and possibly improve healing as well. Post PRK, patients must instill multiple eye-drops of steroids and broad-spectrum antibiotics, and in some cases additional drugs. Most of these must be applied multiple times each day, which could result in poor compliance. The problem of low bioavailability is likely exacerbated during post PRK eye drop based therapy due to the presence of the bandage contact lens. The bandage contact lenses are however very important to the healing process and in fact longer wear durations of about 4 days after PRK could lead to better outcomes. The lenses developed here will be usable post PRK to combine the healing benefits of the contact lens with simultaneous release of multiple drugs that will eliminate the need for eye drops, while improving the drug bioavailability to the target.

Microbial keratitis (MK) is a serious ophthalmic infection that can cause visual impairment and even blindness³⁰. The market for MK is large as estimates for annual cases worldwide are about 1.5 to 2 million though this is likely an underestimate due to underreporting in less developed countries^{30,31}. The MK infection is commonly caused by Pseudomonas aeruginosa and Staphylococcus aureus and is commonly treated by the fourth-generation fluoroquinolones, the newest classes of antibiotics used to treat ocular infections³²⁻³⁵. Moxifloxacin is a fourth-generation fluoroquinolone, commonly used by ophthalmologists due to its superior activity against Staphylococcus aureus as well as gram negative organisms compared to earlier fluoroquinolones such as ofloxacin and ciprofloxacin^{36,37}. For this reason, moxifloxacin is often a first-line choice by ophthalmologists for the treatment and prevention of anterior ophthalmic infections. Also, aqueous humor penetration is highest for moxifloxacin followed by gatifloxacin and lowest for ciprofloxacin³⁸. The aqueous humor penetration is important to achieve the minimum inhibitory concentration (MIC₉₅) of the drug which is in the range from 0.047-0.094 for susceptible strains and from 1.5-12 μg/mL for resistant strains^{39,40} of S. Aurelius. The MIC₅₀ for Pseudomonas aeruginosa to moxifloxacin in keratitis isolates is 3.0 μg/mL⁴¹.

Eye drop therapy for treating ocular infections is sub optimal.

Poor compliance with high frequency regimen for treating severe infections: The frequency of drop instillation for MK is high and it is common to prescribe half-hourly drops or hourly drops for the first 24-72 hours including nights with even more frequent drops every 5 min for the first 30 min.⁴²⁻⁴⁴. Subsequently, the frequency decreases to once every 2 or 4 hours. The high frequency is required because drugs instilled as eye drops remain in contact with the epithelia for only about 5 min. due to the rapid tear drainage and only about 1-5% of the drug permeates into the eye during this time^{11,45,46}. Furthermore, drug that diffuses into the cornea and aqueous humor is cleared via aqueous drainage resulting in an exponential decrease in concentration with time. To keep the aqueous humor concentrations at therapeutic values thus requires frequent eye drops. For example, three drop instillations (0, 15, 30 min) resulted in aqueous humor concentrations of 33.4 μg/g⁴⁷ which is sufficiently high for even the resistant strains. The total number of drops for this therapy though could be as high as about 100 over 1-week of treatment. While efficacious, the patients have difficulties in complying with this high frequency application of eye drops resulting in poor prognosis⁹.

Development of resistance: Antibiotics are becoming less effective because of development of resistance in organisms⁴⁸ to second- and third generation and even the newer fourth-generation fluoroquinolones, such as moxifloxacin^{41,49,50}. There are many factors that could be leading to proliferation of resistant strains including exposure to an insufficient concentration of a bactericidal agent during therapy⁵¹. Any lack of compliance with the high frequency instillation could lead to lower concentrations for the period of noncompliance which could drive development of resistance. Also, there are multiple indications for which recommended dosing is less frequent. For example, the prophylactic dosing of antibiotics after cataract surgery is once every four hours to prevent occurrence of endophthalmitis and dosing of Vigamox (0.5% moxifloxacin) for the treatment of bacterial conjunctivitis is one drop 3 times a day for 7 days⁵². These delivery regimens result in considerably lower aqueous concentrations compared to the MK regimen. For example, instilling drops every 2 hours and every six hours results in maximum aqueous chamber levels of 2.28±1.23 and 0.88±0.88 μg/mL, respectively⁴⁰. These maximum concentrations are higher than MIC for susceptible strains but lower than MIC for resistant strains. Furthermore, the concentration in aqueous humor will decay in the time between successive drops to below the MIC due to the aqueous outflow which could be a factor in development of resistance. Thus, delivery via eye drops is sub optimal for treating infections and low bioavailability of eye drops could be a factor in the disease progression as well as development of antibiotic resistant strains.

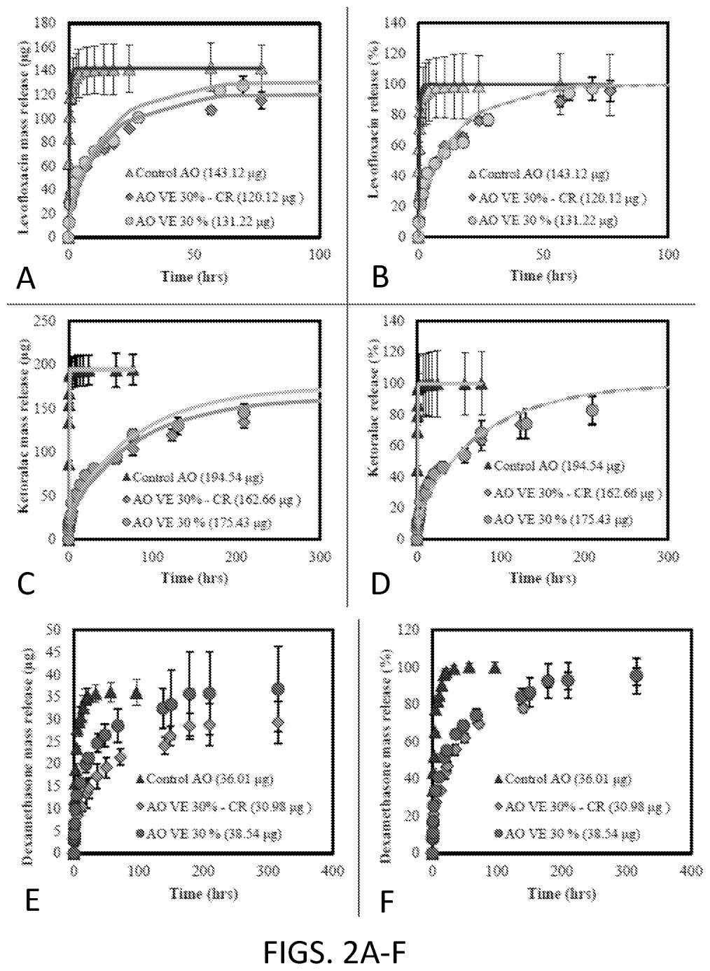

Contact lenses have at least 10-fold higher bioavailability for drug delivered to the aqueous humor compared to eye drops based on our prior research¹². The corneal bioavailability for contact lenses is much higher than drops due to an increase in drug residence time when delivered via contacts²⁶⁻²⁸. The data in FIG. 2A-2F shows that aqueous humor levels are much higher for drug (pirfenidone) delivered via contacts¹² compared to eye drops⁵³, even though dose is 2.5-fold lower in contacts. Contact lens-based drug delivery to treat MK and possibly other indications as well (conjunctivitis, post cataract management) will offer five significant advantages over eye drops: 1. The elimination of frequent eye drop instillations will improve compliance; 2. The high bioavailability will allow concentrations in cornea and aqueous humor to be sufficiently high to treat even the resistant strains; 3. The higher and more consistent concentrations will minimize the possibility of development of resistant strains; 4. The higher concentrations could reduce the time for bacterial eradication; and 5. The higher bioavailability will considerably reduce the systemic exposure. In fact, we will replace the tedious 100-drop therapy that includers instillation of drops at night with just one or two contact lenses which surely be preferable to the patients. It is noted that improper use of contacts can sometimes lead to corneal injury and/or infections, and so typically contact lens use would not be recommended in an infected eye. However, the lenses developed here are drug eluting lenses and so placing these lenses on an infected eye is beneficial because of the improved drug delivery. It is also noted that other researchers⁴⁷ have placed drug-eluting lenses on infected eyes and shown efficacy. It is also noted that the ACUVUE OASYS lenses used here are approved as bandage lenses. Also, we have shown that contact lenses loaded with pirfenidone can lead to healing of cornea after an alkaline burn showing that presence of contact lens is not detrimental to cornea healing¹².

The following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure.

### Example 1

### Experimental Section

### Materials

Commercial ACUVUE® OASYS® lenses (P=−3.50 diopter, BC=8.8 mm) were used for this pilot in vitro study. Ocular medicaments including ketorolac, dexamethasone, levofloxacin (≥98%), and the Tocopherol stereoisomer, Vitamin-E (DL-alpha tocopherol, >96%) were purchased from Sigma-Aldrich. Phosphate buffered saline (PBS 1×), without calcium and magnesium was purchased from Mediatech, Inc. (Manassas, VA, USA. Ethanol (200 proof) was purchased from Decon Laboratories Inc. (King of Prussia, PA, USA). All chemicals were used as received without further purification. Quartz cuvettes used for UV-vis measurements were purchased from Science Outlet.

### Vitamin-E Loading into Commercial Lenses

The commercial ACUVUE® OASYS® lenses were isolated from the blister packs and immersed in de-ionized water for 30 minutes to flush out constituents of the packaging solution. The cleansed lenses were isolated PBS medium with a sterile tweezer and blotted with a Kimwipe to remove residual solution from the lenticular surface. Vitamin-E (VE) was loaded into the hydrogel phase by soaking the lenses in 3 mL of vitamin-E/ethanol solution for 24 hours [12, 18, 25]. After the loading procedure, gels were isolated from the vials, transferred to a 200 mL de-ionized water reservoir, where a soaking duration of 2 hours ensured clearance of ethanol contained in the hydrogel phase. This ensures direct entrapment of vitamin-E within the hydrogel and formation of high aspect ratio nanobarriers. The lenses were isolated again and rinsed with a quick 200-proof ethanol dip of 3-5 seconds to extract vitamin-E deposits adsorbed on the surface of the lenses. The lenses were later rinsed in PBS to shrink the modified lenses to their pre-deformed shape and stored in PBS medium (3 mL) for further experiments. Vitamin-E loading content in the modified lenses were estimated based on difference in dry weights of the both VE modified and control lenses. Different loadings of vitamin-E were integrated into the control lenses by the same protocol. The loading of vitamin E in lens is five times that in the ethanol solution, so soaking ACUVUE OASYS lenses in 4% vitamin E-ethanol solution results in a loading of 20% (w/w) on dry basis^{54,55}. Higher loadings of as much as 70% based on dry weight of the lens can be achieved by soaking in higher concentrations of vitamin E. A concentration of 17.5% vitamin E in ethanol will yield about 70% vitamin E loading in the lens. The vitamin E barriers are disk-like with an average thickness and radius of 21 and 350 nm, respectivelyl. The diameter of the control Acuvue Oasys lenses (senofilcon A) was 14 mm with a base curvature of 8.4-8.8 mm. The diameter of the control lenses upon vitamin-E modification increased by 10% with no significant changes induced to the base curvature.

### In Vitro Triumvirate Loading of Anti-Microbial, Corticosteroid, and Anti-Microbial Medicaments into Modified Lenses

The commercial ACUVUE® OASYS® lenses were isolated from the PBS medium and blotted with a Kimwipe to remove residual solution from the surface of the lens. Excess dexamethasone was added to 1× PBS and subjected to vortex mixing to facilitate drug dissolution and avoid adhesion of hydrophobic drug particulate to the inner surface of the control vial. The prepared drug solution was later centrifuged to obtain a supernatant of 0.07 mg/mL dexamethasone/PBS solution. Solid drug particulates of levofloxacin and ketorolac were added to the supernatant dexamethasone/PBS solution to yield concentrations of 25 mg/mL and 5 mg/mL respectively. The control ACUVUE® OASYS® lenses were soaked in 3 mL of the drug mixture/PBS solution per lens for uptake measurements. The dynamic concentration of the drugs in aqueous phase were not monitored during the drug loading phase to quantify the partitioned drug owing to high magnitude of experimental absorbance values. The vitamin-E modified lenses were immersed in a batch of 3 mL of drug solution for prolonged duration up to a month. After the loading phase, the lenses were taken out and excess drug solution was blotted from the surface of the lens and subsequently used for release experiments.

### In Vitro Triumvirate Drug Release from Vitamin-E Modified Lenses

The release studies of the three tested drugs (Levofloxacin, Ketorolac, and Dexamethasone) from 30 w/w % vitamin-E modified ACUVUE® OASYS® lenses were done through lens immersion in PBS medium. The drug laden lenses were isolated from the loading solutions and blotted with a Kimwipe to remove residual solution from the lenticular surface. The volume ratio of both the phases for release studies in PBS was fixed at 100.

### Transmittance of Control, Vitamin-E, and Drug Incorporated Commercial Lenses

Transmittance spectral measurements were obtained by a UV-Vis spectrophotometer (GENESYS™ 10 UV, Thermo Spectronic, Rochester, NY, USA) equipped with detecting transmittance of the soft contact lenses in the spectral range of 190 nm-1100 nm. The soft contact lenses (ACUVUE® OASYS® and ACUVUE® TruEye™ lenses) soaked prior to obtaining a transmittance spectrum, were taken out of the PBS medium and blotted with a Kimwipe to remove residual solution on the surface of the lenses. The dried hydrogel was carefully mounted on the outer surface of the quartz cuvette without inducing structural damage to the hydrogel material. The outer surface of the cuvette chosen for affixing the hydrogel was a region visible through the cell holder's aperture to allow exposure to a monochromatic ultra-violet (UV) beam for recording the transmittance spectra. The transmittance measurements shown in FIG. 1 were taken at a 1 nm interval in the spectral bandwidth of 190 nm-1100 nm. The spectral bandwidth of 190 nm-1100 nm was chosen to gauge the effect of vitamin-E incorporation on the transmittance of the modified lenses. Vitamin-E incorporation into commercial ACUVUE® OASYS® and ACUVUE® TruEye™ lenses does not affect the material's ability to transmit visible light in the range of 500 nm-700 nm. No structural changes or loss of transparency was observed after vitamin-E incorporation as shown in FIG. 1.

### Water Content of Commercial Lenses Post-Vitamin-E and Drug Incorporation

The percentage water content in the commercial lenses was determined through differences in mass of hydrated and dry gels. The hydrated gels were stored in PBS medium prior to measurements and later dried at 60° C. in a convection oven to obtain dry gel mass. The equilibrium water content was evaluated as

$$\% WC = ( \frac{{W}_{hydrated} - {W}_{dry}}{{W}_{hydrated}} ) \times 100 \qquad ( 1 )$$

Table 1 summarizes the water content of control ACUVUE® OASYS® and ACUVUE® TruEye™ lenses and the change observed after incorporation of vitamin-E in the lens matrix. A 2-16% reduction in water content of the hydrogels depending on the lens type is observed due to integration of hydrophobic vitamin-E aggregates in the polymer matrix.

### Ion Permeability of Commercial Lenses Post-Vitamin-E and Drug Incorporation

The permeability of contact lenses is critical to maintain ion and oxygen transport across the cornea. The former is critical for maintenance of lens motion while the latter is necessary for constant supply to the avascular cornea. The recommended ion permeability of the silicone-based lenses is at least 2×10⁻⁶ mm²/min to allow sufficient lens motion and ion transport to prevent corneal adherence and hypoxia [27]. The ion permeability of the control and modified lenses were estimated through dynamic salt concentration measurements in perfect sink conditions. Commercial lenses of ˜30 μL gel volume were initially soaked in 5 mL sodium chloride/water solutions of concentrations ranging from 0.5 M-1 M at room temperature (25° C.) overnight. A period of 12 hours was chosen to allow salt equilibration between the two phases. The salt loaded lenses were then soaked in a 30 mL aqueous medium (water) and dynamic ionic conductivity of released salt was determined through a Con 450 probe (OAKTON). The measured ionic conductivity was related to salt concentration through a linear calibration curve with a slope of 4×10⁻⁵ M/μS. The calibration curve was obtained through conductivity measurements of salt solutions of concentrations ranging from 0.5 M-1 M. The volume ratio of release medium to gel phase was fixed at 1000 to satisfy the perfect sink condition and the salt partition coefficient was estimated by

$$K = \frac{{V}_{w} {C}_{r}}{{V}_{l} {C}_{l}} \qquad ( 2 )$$

where Vₗ and V_{w} are the volumes of lens and fluid in release medium and Cₗ and Cᵣ are the starting concentration during loading and final concentration in release, respectively. By fitting the experimental concentration data with a simple diffusion control model, the salt diffusivity from the commercial lenses was estimated. Ion permeability of the lenses was then estimated through experimental partition coefficient values and salt diffusivities from the fits. Table 2 summarizes the material transport properties that dictate lens motion and ion supply to the cornea. The estimated permeability values indicate that incorporation of vitamin-E in the lens matrix is at least two orders of magnitude higher than the recommended ion flux permeability. Interestingly, upon vitamin-E incorporation, a minor decrease in ion permeability is observed.

### In Vitro Model

To obtain the transport properties of the drugs, the in vitro data was fit a one-dimensional Fickian diffusion model. Owing to high hydrophilicity of levofloxacin and ketorolac and disparity in volume of the reservoir and the lens, perfect sink conditions were assumed to evaluate the distribution coefficients of these drugs in the hydrogel phase. The following expression was utilized to evaluate the partition coefficient of the drugs:

$$K = \frac{{V}_{w} {C}_{r}}{{V}_{l} {C}_{l}} \qquad ( 3 )$$

where Vₗ and V_{w} are the volumes of lens and fluid in release medium and Cₗ and Cᵣ are the starting concentration during loading and final concentration in release, respectively.

On the other hand, the release of dexamethasone is not under sink conditions which implies that only a limited amount of drug will diffuse out till equilibrium is achieved. In such cases the partition coefficient can be calculated by solving the following implicit equation,

$${C}_{r} = \frac{{KC}_{l} {V}_{w , l} {V}_{g}}{( {V}_{w , l} + {KV}_{l} ) ( {V}_{w , r} + {KV}_{l} )}$$

where V_{w,l} and V_{w,r} are the fluid volumes during loading and release, respectively.

The drug transport in the transverse direction can be described as:

$$\frac{\partial {C}_{l}}{\partial t} = {D}_{l} ( \frac{{\partial }^{2} {C}_{l}}{\partial {y}^{2}} ) , \qquad ( 7 )$$

where Cₗ is the drug concentration in the contact lens and Dₗ is drug diffusivity in the lens. The boundary and initial conditions for diffusion in the lens matrix are:

$$\frac{\partial {C}_{l}}{\partial y} ( t , y = 0 ) = 0 \qquad ( 8 )$$

$${C}_{l} ( t , y = {h}_{l} ) = {KC}_{w} ( t ) \qquad ( 9 )$$

$${C}_{l} ( t = 0 ) = 0 , \qquad ( 10 )$$

where h_{g} is the half-thickness of the lens (˜40 μm for commercial ACUVUE® OASYS® and ACUVUE® TruEye™ lenses). The boundary condition (8) arises from symmetry of the hydrogel matrix and that in (9) assumes equilibrium between concentration of the drug in the gel matrix and the surrounding formulation present in the aqueous reservoir in the vial (˜3 mL). For hydrophilic drugs, owing to low distribution coefficient in the hydrogel phase, drug concentration in the gel's leading edge reduces to zero. To account for the loss of drug concentration in the reservoir due to its influx into the secondary gel phase, a mass balance on the aqueous Levofloxacin and Ketoralac reservoir in the scintillation vial yields the following equation:

$${V}_{w} \frac{{dC}_{w}}{dt} = - 2 {D}_{l} {A}_{l} \frac{\partial {C}_{l}}{\partial y} ( y = {h}_{g} ) , \qquad ( 11 )$$

where V_{w} is the volume of solution in the aqueous reservoir and Aₗ is the area of contact between the lens and the fluid on either edge. The modelled diffusion equation was solved using finite difference schemes in MATLAB.

### In Vitro Studies—Dynamics of Triumvirate Drug Transport from Control and Vitamin-E Modified Lenses

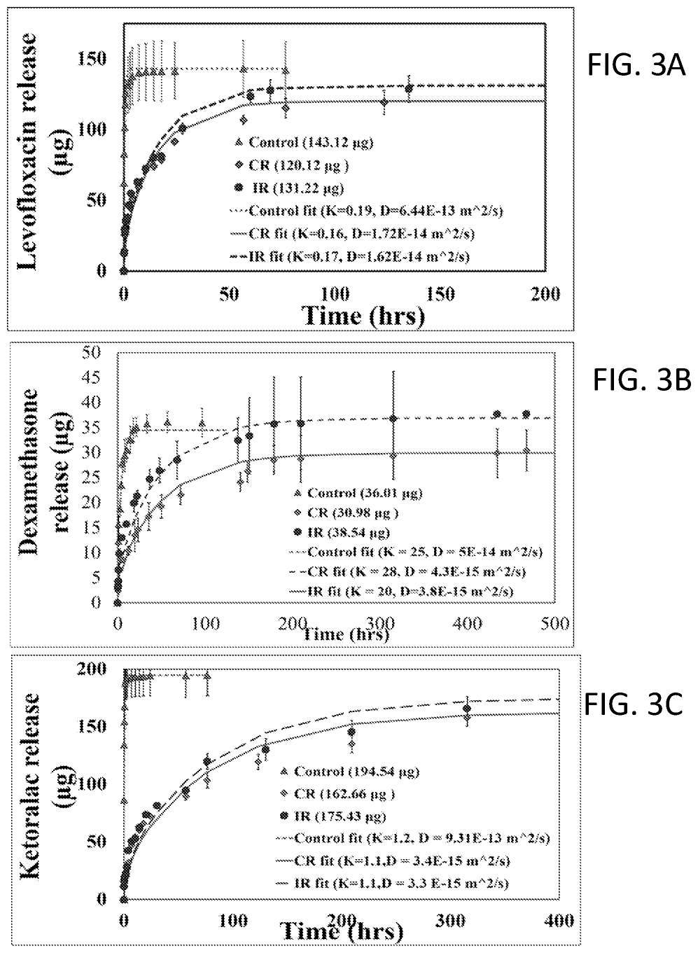

FIG. 3A-3C shows that lenses with 30% vitamin E release of dexamethasone, ketorolac and levofloxacin to 200, 200, 60 hours compared to a few hours for the control lens. The release can be tuned to the target by changing the vitamin E loading. The release of each of the drug loaded separately (IR: individual release) is comparable to the release of the same drug when loaded with the other two drugs (CR). Drug was loaded in lenses by soaking in 80 ug dexamethasone, 5 mg/mL ketorolac and 25 mg/mL levofloxacin. Solid lines represent fits to the data based on diffusion control release. The model fits the data well with best fit diffusivity values and partition coefficients included in the legends.

### In Vivo Model for Predicting the Corneal Bioavailability of Levofloxacin and Ketorolac

The contact lens releases drug into the post-lens tear film or the pre-lens tear films. The drug released into the post lens tear film can diffuse through the epithelium and then through the stroma and endothelium to reach the aqueous humor. To predict the bioavailability of drugs delivered by contact lens based therapy, a simplified version of in vivo model developed by Li & Chauhan, 2006 will be used²⁷. The governing diffusion model predicts the mass of drug partitioned into the POLTF domain upon lens wear and its subsequent uptake into the eye. In this simplified model, the drug laden hydrogel lens is treated as a thin, two-dimensional polymer network with area A_{cont} and half-thickness h. The rigid and stationary cornea is shielded by a hydrogel lens whose motion is driven by the tear film lake during the drug release phase. Axial dispersion driven by blinking combination of shear and squeeze flow is neglected. With fractional drug loss attributed to POLTF dispersion shown to be <5%, it is reasonable to ignore its contribution and treat drug concentration in the POLTF domain to be spatially independent. The corneal curvature (˜1.2 cm) is also ignored due to disparity in length scales in comparison to the tear film thickness (˜10 μm). To determine the mass of drugs delivered to the aqueous humor, the governing gel diffusion along with the mass balances in the POLTF and aqueous humor must be solved along with the appropriate initial and boundary conditions.

The drug transport in the contact lens, post-lens tear film and aqueous humor can be described as:

$$\frac{\partial {C}_{l}}{\partial t} = {D}_{l} \frac{{\partial }^{2} {C}_{l}}{\partial {y}^{2}} \\ {V}_{t} \frac{{dC}_{t}}{dt} = - {D}_{l} \frac{\partial {C}_{l}}{\partial y} ( y = 2 h ) {A}_{l} - {A}_{cornea} {k}_{cornea} ( {C}_{t} - {C}_{aq} ) \\ {V}_{aq} \frac{\partial {C}_{aq}}{\partial t} = {A}_{cornea} {k}_{cornea} ( {C}_{t} - {C}_{aq} ) - {q}_{aq} {C}_{aq} ,$$

where Cₜ and C_{aq} are the time dependent concentrations in the POLTF and aqueous humor, respectively, and Cₗ is the time and position dependent concentration in the contact lens, Vₜ and Vₜ are the volumes of aqueous humor and POLTF, respectively, both of which are treated as well-mixed, Aₗ and A_{cornea} are the areas of the lens and cornea, respectively, Dₗ is drug diffusivity in lens, k_{cornea} is permeability of cornea to the drug, and q_{aq} is the rate of drainage of the aqueous humor. Since Eq. (1) is a partial differential equation, it requires the following boundary conditions, Cₗ(y=2h)=KₗCₜ, which implies equilibrium between tears and lens and Cₗ(y=0)=0, which implies that the drug released by the lens towards the pre-lens tear film is rapidly carried away via tear drainage resulting in negligible concentration in the pre-lens tear film. Finally, at t=0, all tissue concentrations are zero and the concentration in the anterior or posterior contact lens is equal to the loading concentration (C_{l,i}). In a further simplification of the model, the drug release can be under sink conditions, under which we obtain the following expression for the concentration in the aqueous humor.

$${C}_{aq} = {\sum }_{n = 0}^{∞} {C}_{l , i} \frac{2 DA / h}{{q}_{aq} - \frac{{( 2 n + 1 )}^{2} {π}^{2}}{4 {h}^{2}} {DV}_{aq}} [ {e}^{- \frac{{( 2 n + 1 )}^{2} {π}^{2}}{4 {h}^{2}} Dt} - {e}^{- \frac{{q}_{aq}}{{V}_{aq}} t} ]$$

This sink model was developed and validated for hydrophilic drugs such as pirfenidone with sufficiently highly corneal permeability such that the time scale for drug released into the post-lens tear film is shorter than the time scale for transport into the cornea¹⁴. This simplified model is likely valid for levofloxacin and ketorolac as well because both are hydrophilic of comparable size to pirfenidone. However, the non-sink model proposed above is likely a more accurate predictor for hydrophobic drug dexamethasone. Eye drops. A model for eye drops under similar assumptions gives the following expression for aqueous humor concentration after instillation of eye drops,

$${C}_{aq} = \sum \frac{{fC}_{drop} {V}_{drop}}{{V}_{aq}} {e}^{- \frac{{q}_{aq}}{{V}_{aq}} ( t - {t}_{i} )} H ( ( t - {t}_{i} ) ,$$

where each term in the summation represents from the drop delivered at time tᵢ, C_{drop} (=0.5%) and V_{drop} (=30 microliter) are the volume and drug concentration in the eyedrops, f is the bioavailability that ranges from 1-5%³ and H((t−tᵢ) is the Heavyside function that is 1 for t>tᵢ and 0, otherwise. The eye drop model was validated by comparing the aqueous humor concentrations after instillation of pirfenidone eye drops^{14,56}. Levine et al. reported that the concentration with cataract prophylaxis dosing (4× daily), the mean aqueous concentration of moxifloxacin was 1.745 μg/mL (range 0.92 to 3.87 μg/mL)⁵⁷ compared to the model prediction of 2.8 μg/mL. The predictions are higher than measurements suggesting lower bioavailability than the assumed value of 1%.

DEXTENZA® (0.4 mg) is a corticosteroid intracanalicular insert that released drug int o the tears for about 30 days. The tear concentration after insertion of DEXTENZA (C_{t,Dextenza}) has been reported and based on that the concentration of drug in the aqueous humor can be estimated by equating the rate of transport from tears to the aqueous humor and the rate of aqueous clearance. Based on this we get the following expression,

$${C}_{aq} = \frac{{k}_{cornea} {C}_{t , Dextenza}}{{q}_{aq}} .$$

DEXYCU® 9% (Dose_{Dexycu}=0.5 mg) releases the drug for about 30 days (=T_{Dexycu}). The formulation is injected behind the iris in the inferior portion of the posterior chamber. Assuming a zero order release the concentration in aqueous humor will be given by the following expression,

$${C}_{aq} = \frac{{Dose}_{Dexycu}}{{q}_{aq} {T}_{Dexycu}} .$$

The same model is used to model Surodex (60 μg), another dexamethasone releasing insert⁵⁸.

Intracameral (IC) antibiotics⁵⁹ provides an impulse of drug (Dose) that is than cleared via aqueous outflow giving the following expression for concentration,

$${C}_{aq} = \frac{{Dose}_{IC}}{{V}_{aq}} {e}^{- \frac{{q}_{aq} t}{{V}_{aq}}}$$

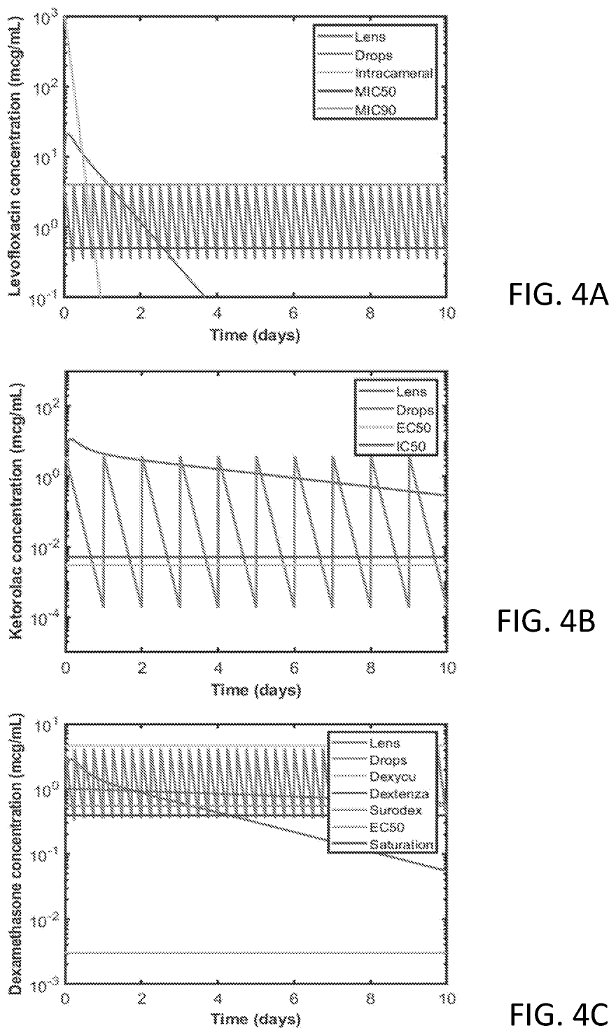

FIGS. 4A-4C shows the predicted concentrations in aqueous humor for eye drop or lens-based therapies in New Zealand white rabbit based on the model summarized above and validated in our previous work¹⁴. The values of model parameters listed in Table 1 are obtained or estimated based on literature^{60,25,27,3}. The figure also includes predictions for Surodex, DEXYCU® and DEXTENZA and reported values of minimum inhibitory concentration (MIC₅₀ and MIC₉₀ for pseudomonas aeruginosa) for levofloxacin⁶¹, Effective concentration (EC) and Inhibitory concentration (IC) for ketorolac^{62,63} and concentrations for 50% (EC50)⁶⁴ and saturation of dexamethasone binding receptors⁶³. The model predictions for contact lens delivery of ketorolac show that the aqueous humor concentration remain comparable to the maximum concentration with drops and considerably higher than the EC50 and IC50 for more than 10 days. The predictions for dexamethasone show that the concentration remains higher than EC50 for more than 10 days, but it is below the window of concentrations with other devices after about 5 days. The predictions for levofloxacin show that concentration with lens drops below MIC50 and the trough of eye drops after about 3 days. Thus, the contact lens could replace the eye drops for about three days.

### Example 2

Moxifloxacin delivery for treating infections. Vitamin E was loaded in the OASYS contact lenses by soaking the lenses in vitamin E dissolved in ethanol at 4 mg/mL. The lenses were then soaked in buffer to extract ethanol resulting in formation of vitamin E barriers. The lenses were then soaked in drug solutions till equilibrium was achieved, followed by soaking in buffer to measure in vitro release.

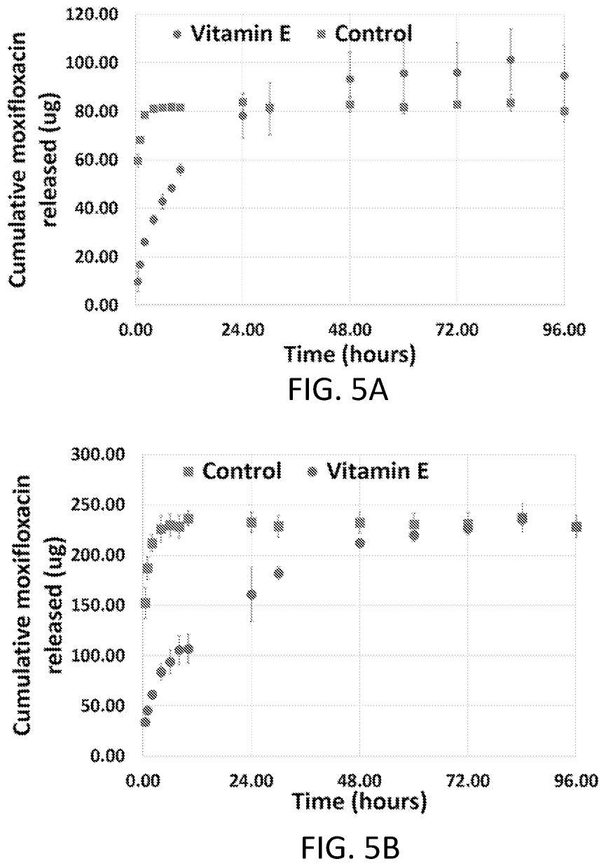

In vitro release in FIGS. 5A-5B shows that 20% VE loaded lenses soaked in 5 and 20 mg/ml of drug solution will release 100 and 225 μg over 3 days. Since vitamin E increase leads to a quadratic increase in release duration, we expect 30% VE lenses to release MOX over 7-days.

### Example 3

In this example we will develop a contact lens with enhanced loading of dexamethasone and antibiotics to have the ability to provide concentrations higher than that with eye drops for 7 days. The physician will have the option of replacing the lens with another lens after 7 days. Increasing the drug loading of dexamethasone is difficult due to the solubility limit so we will instead load dexamethasone sodium phosphate (DXP) into the lenses. Our prior research has shown that ACUVUE OASYS lenses loaded with 30% VE can sustain dexamethasone phosphate (DXP) release for about three weeks and the partition coefficient for DXP in ACUVUE OASYS is about 1.2⁵¹. Based on this data we expect that lenses loaded with 30% VE by soaking in 20 mg/ml of DXP will release about 750 μg of drug in 40 days. Increasing release duration of levofloxacin from 3 days to 7 days will require increasing vitamin E concentration. An alternative is to replace levofloxacin with moxifloxacin which is a commonly used 4^{th} generation fluoroquinolone. Additionally, bromfenac will be incorporated as a replacement for ketorolac. Thus, the triple drug combination will be Moxifloxacin, Dexamethasone, Bromfenac, or Moxifloxacin, Dexamethasone Phosphate, Bromfenac, or Moxifloxacin, Dexamethasone, Ketorolac or Moxifloxacin, Dexamethasone Phosphate, Ketorolac. Any of these drugs can be substituted with drugs in the same general class. Additionally, any one of two of these drugs could be loaded instead of all three. For example, just a steroid and NSAID could be loaded and the antibiotic could be administered as an intracameral injection. It should also be understood that the drugs listed above are typically used as the salt form. For example, moxifloxacin hydrochloride, ketorolac tromethamine, bromfenac sodium.

### Example 4

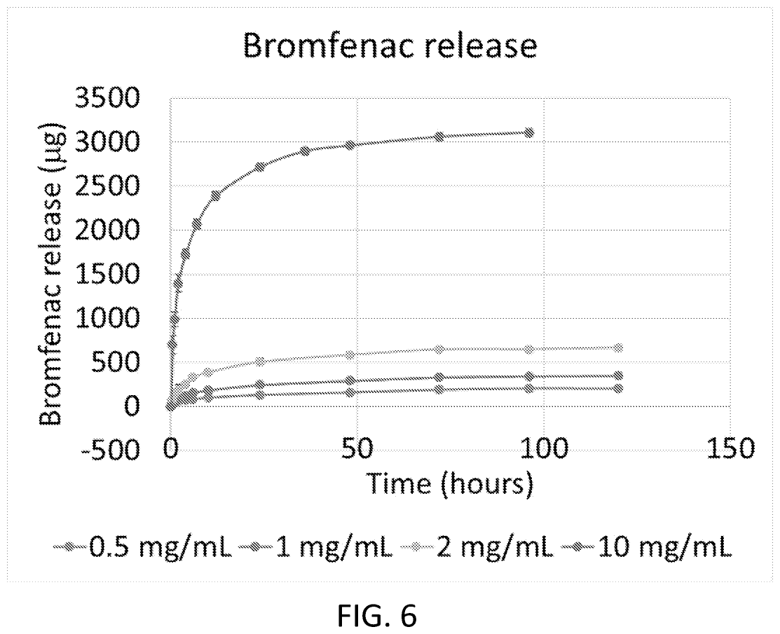

Delivery of bromfenac was tested by loading the drug in Acuvuce Oasys with 20% vitamin E lenses in a 3 mL solution of bromfenac at 0.5, 1, 2 and 10 mg/mL (as shown in FIG. 6). After equilibrium was achieved in 7 days, lenses were soaked in 3 mL of buffer to measure drug release rates and also calculate the partition coefficient, i.e, the ratio of drug concentration in lens and solution. The data shows that the vitamin E loaded lens sustains bromfenac release for about 3 days and the partition coefficient is 11.65±1.28. The release duration can be increased to about 7-days by increasing vitamin E loading in lens to about 30% because the release duration increases as square of the vitamin E loading. The high partition coefficient means that a concentration of just 1 mg/mL in loading solution will be adequate to load about 350 μg of drug.

It should be emphasized that the above-described embodiments of the present disclosure are merely possible examples of implementations set forth for a clear understanding of the principles of the disclosure. It should also be emphasized that models used for in vivo predictions are uncertain and are used only as guides for developing optimal contact lenses for achieving desired concentrations. Many variations and modifications may be made to the above-described embodiment(s) without departing substantially from the spirit and principles of the disclosure. All such modifications and variations are intended to be included herein within the scope of this disclosure and protected by the following claims.

TABLE 1
Summary of water content of control and vitamin-E modified
ACUVUE ® OASYS ® lenses.

ACUVUE ® OASYS ®

| VE loading (aq. phase) (%) | 0 | 10 | 20 |
|---|---|---|---|
| Soaking duration in PBS (hr) | 24 | 24 | 24 |
| Hydrated weight (mg) | 31.9 ± 0.5 | 37.0 ± 2.7 | 36 |
| Dry weight (g) | 19.8 ± 0.3 | 24.3 ± 0.2 | 24.5 |
| Water content (%) | 37.9 ± 0.2 | 34.2 ± 0.9 | 31.8 |
| Number of experiments | 3 | 3 | 1 |

TABLE 2
Summary of ion permeability of control and vitamin-E modified
ACUVUE ® OASYS ® lenses.

| | ACUVUE ® OASYS ® | | |
|---|---|---|---|
| VE loading (aq. phase) (%) | 0 | 10 | 20 |
| Loading duration (hr) | 24 | 24 | 24 |
| Partition Coefficient (K) | 0.82 ± 0.05 | 0.95 ± 0.17 | 1.02 |
| Salt Diffusivity | 0.802 | 0.53 | 0.11 |
| (×10⁻⁴ mm²/min) | | | |
| Permeability | 0.65 ± 0.04 | 0.50 ± 0.1 | 0.11 |
| (×10⁻⁴ mm²/min) | | | |
| Number of experiments | 3 | 3 | 1 |

TABLE 3
Parameters used for the in vivo and non-perfect sink diffusion
models. Permeabilities obtained from Reference.⁵⁶

| Parameter | Description | Values | |
|---|---|---|---|
| A_{cornea} | Corneal Surface Area | 104 | mm² |
| Aₗₑₙₛ | Lenticular Surface Area | 230 | mm² |
| Vₜₑₐᵣ | POLTF volume | 2.3 | μL |
| h_{g} | Half-Thickness of Lens | 50 | μm |

| | | |
|---|---|---|
| K_{cornea, levofloxacin} | Corneal permeability of | 1.2 × |
| | levofloxacin | 10⁻⁵ cm/s |
| K_{cornea, ketorolac} | Corneal permeability of | 2.5 × |
| | ketorolac | 10⁻⁵ cm/s* |
| K_{cornea, dexamethasone} | Corneal permeability of | 1.2 × |
| | dexamethasone | 10⁻⁵ cm/s* |

*Assumed to be equal to that for timolol

### REFERENCES

1. Peng, C. C., Burke, M. T. & Chauhan, A. Transport of topical anesthetics in vitamin E loaded silicone hydrogel contact lenses. Langmuir 28, 1478-1487 (2011).
2. Foster, A. Vision 2020: The cataract challenge. Journal of Community Eye Health vol. 13 17-19 (2000).
3. Dick, H. B., Schwenn, O., Krummenauer, F., Krist, R. & Pfeiffer, N. Inflammation after sclerocorneal versus clear corneal tunnel phacoemulsification. Ophthalmology 107, 241-247 (2000).
4. Zimmerman, T. J. & Zalta, A. H. Facilitating patient compliance in glaucoma therapy. Surv. Ophthalmol. 28, 252-258 (1983).
5. Ashburn, F. S., Goldberg, I. & Kass, M. A. Compliance with ocular therapy. Surv. Ophthalmol. 24, 237-248 (1980).
6. Kholdebarin, R., Campbell, R. J., Jin, Y. P., Buys, Y. M., Beiko, G., Birt, C., Damji, K., Feldman, F., Lajaoie, C., Kulkarni, S., Martow, J., Sakamoto, T., Spencer, J. & Trope, G. E. Multicenter study of compliance and drop administration in glaucoma. Can. J. Ophthalmol. 43, 454-461 (2008).
7. Tsai, J. C., McClure, C. A., Ramos, S. E., Schlundt, D. G. & Pichert, J. W. Compliance barriers in glaucoma: A systematic classification. J. Glaucoma 12, 393-398 (2003).
8. MacKean, J. M. & Elkington, A. R. Compliance with treatment of patients with chronic open-angle glaucoma. Br. J. Ophthalmol. 67, 46-49 (1983).
9. Winfield, A. J., Jessiman, D., Williams, A. & Esakowitz, L. A study of the causes of non-compliance by patients prescribed eyedrops. Br. J. Ophthalmol. 74, 477-480 (1990).
10. Bron, A., Hermann, M. & Creuzot-Garcher, C Diestelhorst, M. Monitoring individual compliance in glaucoma patients used to topical therapy. in Program and abstracts of the Association for Research in Vision and Ophthalmology 2007 Annual Meeting (2007).
11. Agrahari, V., Mandal, A., Agrahari, V., Trinh, H. M., Joseph, M., Ray, A., Hadji, H., Mitra, R., Pal, D. & Mitra, A. K. A comprehensive insight on ocular pharmacokinetics. Drug Deliv. Transl. Res. 6, 735-754 (2016).
12. Dixon, P., Ghosh, T., Mondal, K., Konar, A., Chauhan, A. & Hazra, S. Controlled delivery of pirfenidone through vitamin E-loaded contact lens ameliorates corneal inflammation. Drug Deliv. Transl. Res. 8, 1114-1126 (2018).
13. Liu, Y.-C., Wong, T. T. & Meta, J. S. Intraocular lens as a drug delivery reservoir. Curr. Opin. Ophthalmol. 24, 53-59 (2013).
14. Marcel, M., Can, H. ⋅ & Diestelhorst, M. Electronic compliance monitoring of topical treatment after ophthalmic surgery. doi: 10.1007/s10792-010-9362-3.
15. An, J. A., Kasner, O., Samek, D. A. & Lévesque, V. Evaluation of eyedrop administration by inexperienced patients after cataract surgery. J. Cataract Refract. Surg. 40, 1857-1861 (2014).
16. Dietlein, T. S., Jordan, J. F., Lüke, C., Schild, A., Dinslage, S. & Krieglstein, G. K. Self-application of single-use eyedrop containers in an elderly population: Comparisons with standard eyedrop bottle and with younger patients. Acta Ophthalmol. 86, 856-859 (2008).
17. Taban, M., Sarayba, M. A., Ignacio, T. S., Behrens, A. & McDonnell, P. J. Ingress of India ink into the anterior chamber through sutureless clear corneal cataract wounds. Arch. Ophthalmol. 123, 643-648 (2005).
18. Shorstein, N. H. & Myers, W. G. Drop-free approaches for cataract surgery. Curr. Opin. Ophthalmol. 31, 67-73 (2020).
19. Donnenfeld, E. & Holland, E. Dexamethasone Intracameral Drug-Delivery Suspension for Inflammation Associated with Cataract Surgery: A Randomized, Placebo-Controlled, Phase III Trial. Ophthalmology 125, 799-806 (2018).
20. Kiernan, D. F. Dexamethasone intracameral drug-delivery suspension for inflammation associated with vitreoretinal surgery. BMJ Open Ophthalmol. 5, 491 (2020).
21. Donnenfeld, E. D., Solomon, K. D. & Matossian, C. Safety of IBI-10090 for inflammation associated with cataract surgery: Phase 3 multicenter study. J. Cataract Refract. Surg. 44, 1236-1246 (2018).
22. Song, H., Li, Y., Zhang, Y., Shi, D. & Li, X. Bandage Lenses in the Postoperative Care for Cataract Surgery Patients: A Substitute for Eye Patch? J. Ophthalmol. 2018, (2018).
23. Debasish, M. & Debabrata, B. Comparison of safety and efficacy of drug delivery by topical application versus drug-eluting contact lens in cataract surgery. Indian Journal of Ophthalmology vol. 69 466-467 (2021).
24 Shi, D. N., Song, H., Ding, T., Qiu, W. Q. & Wang, W. Evaluation of the safety and efficacy of therapeutic bandage contact lenses on post-cataract surgery patients. Int. J. Ophthalmol. 11, 230-234 (2018).
25. Zhu, H. & Chauhan, A. A mathematical model for ocular tear and solute balance. Curr. Eye Res. 30, 841-854 (2005).
26 Creech, J. L., Chauhan, A. & Radke, C. J. Dispersive mixing in the posterior tear film under a soft contact lens. Ind. Eng. Chem. Res. 40, (2001).
27. McNamara, N. A., Polse, K. A., Brand, R. J., Graham, A. D., Chan, J. S. & McKenney, C. D. Tear mixing under a soft contact lens: Effects of lens diameter. Am. J. Ophthalmol. 127, 659-665 (1999).
28. Li, C. C. & Chauhan, A. Modeling ophthalmic drug delivery by soaked contact lenses. Ind. Eng. Chem. Res. 45, 3718-3734 (2006).
29 Zhu, H. & Chauhan, A. Tear dynamics model. Curr. Eye Res. 32, 177-197 (2007).
30. Ung, L., Bispo, P. J. M., Shanbhag, S. S., Gilmore, M. S. & Chodosh, J. The persistent dilemma of microbial keratitis: Global burden, diagnosis, and antimicrobial resistance. Surv. Ophthalmol. 64, 255-271 (2019).
31. Whitcher, J. P., Srinivasan, M. & Upadhyay, M. P. Corneal blindness: A global perspective. Bull. World Health Organ. 79, 214-221 (2001).
32 Daniell, M., Mills, R. & Morlet, N. Microbial keratitis: what's the preferred initial therapy? Ophthalmology 87, 1167 (2003).
33. Robertson, D. M., Rogers, N. A., Petroll, W. M. & Zhu, M. Second harmonic generation imaging of corneal stroma after infection by Pseudomonas aeruginosa. Sci. Rep. 7, 1-10 (2017).
34. Mather, R., Karenchak, L. M., Romanowski, E. G. & Kowalski, R. P. Fourth generation fluoroquinolones: New weapons in the arsenal of ophthalmic antibiotics. Am. J. Ophthalmol. 133, 463-466 (2002).
35. Chaudhry, N. A., Flynn, H. W., Murray, T. G., Tabandeh, H., Mello, M. O. & Miller, D. Emerging ciprofloxacin-resistant Pseudomonas aeruginosa. Am. J. Ophthalmol. 125, 509-510 (1999).
36. Thibodeaux, B. A., Dajcs, J. J., Caballero, A. R., Marquart, M. E., Girgis, D. O. & O'Callaghan, R. J. Quantitative comparison of fluoroquinolone therapies of experimental Gram-negative bacterial keratitis. Curr. Eye Res. (2004) doi: 10.1076/ceyr.28.5.337.28676.
37. Dajcs, J. J., Thibodeaux, B. A., Marquart, M. E., Girgis, D. O., Traidej, M. & O'Callaghan, R. J. Effectiveness of ciprofloxacin, levofloxacin, or moxifloxacin for treatment of experimental Staphylococcus aureus keratitis. Antimicrob. Agents Chemother. 48, 1948-1952 (2004).
38. Solomon, R., Donnenfeld, E. D., Perry, H. D., Snyder, R. W., Nedrud, C., Stein, J. & Bloom, A. Penetration of topically applied gatifloxacin 0.3%, moxifloxacin 0.5%, and ciprofloxacin 0.3% into the aqueous humor. Ophthalmology 112, 466-469 (2005).
39 Kowalski, R. P., Yates, K. A., Romanowski, E. G., Karenchak, L. M., Mah, F. S. & Gordon, Y. J. An ophthalmologist's guide to understanding antibiotic susceptibility and minimum inhibitory concentration data. Ophthalmology 112, 16183128 (2005).
40. Miller, D. Review of moxifloxacin hydrochloride ophthalmic solution in the treatment of bacterial eye infections. Clin. Ophthalmol. 2, 77-91 (2008).
41. Oldenburg, C. E., Lalitha, P., Srinivasan, M., Rajaraman, R., Ravindran, M., Mascarenhas, J., Borkar, D. S., Ray, K. J., Zegans, M. E., McLeod, S. D., Porco, T. C., Lietman, T. M. & Acharya, N. R. Emerging moxifloxacin resistance in pseudomonas aeruginosa keratitis isolates in South India. Ophthalmic Epidemiol. 20, 155-158 (2013).
42. Austin, A., Lietman, T. & Rose-Nussbaumer, J. Update on the Management of Infectious Keratitis. Ophthalmology 124, 1678-1689 (2017).
43. Gangopadhyay, N., Daniell, M., Weih, L. A. & Taylor, H. R. Fluoroquinolone and fortified antibiotics for treating bacterial corneal ulcers. Br. J. Ophthalmol. 84, 378-384 (2000).
44. Gokhale, N. S. Medical management approach to infectious keratitis. Indian J. Ophthalmol. 56, 215-220 (2008).
45. Jünemann, A. G. M., Chora̧giewicz, T., Ozimek, M., Grieb, P. & Rejdak, R. Drug bioavailability from topically applied ocular drops. Does drop size matter? Ophthalmol. J. 1, 29-35 (2016).
46. Urtti, A. & Salminen, L. Minimizing systemic absorption of topically administered ophthalmic drugs. Surv. Ophthalmol. 37, 435-456 (1993).
47. Kakisu, K., Matsunaga, T., Kobayakawa, S., Sato, T. & Tochikubo, T. Development and efficacy of a drug-releasing soft contact lens. Investig. Ophthalmol. Vis. Sci. 54, 2551-2561 (2013).
48. Schulte, A. J., Agan, B. K., Wang, H. C., McGann, P. T., Davies, C. B. W., Legault, G. L. & Justin, G. A. Multidrug-resistant organisms from ophthalmic cultures: Antibiotic resistance and visual acuity. Mil. Med. 185, e1002-e1007 (2020).
49. Kunimoto, D. Y., Sharma, S., Garg, P. & Rao, G. N. In vitro susceptibility of bacterial keratitis pathogens to ciprofloxacin: Emerging resistance. Ophthalmology 106, 80-85 (1999).
50. Moshirfar, M., Mirzaian, G., Feiz, V. & Kang, P. C. Fourth-generation fluoroquinolone-resistant bacterial keratitis after refractive surgery. J. Cataract Refract. Surg. 32, 515-518 (2006).
51. Stratton, C. W. Dead bugs don't mutate: Susceptibility issues in the emergence of bacterial resistance. Emerg. Infect. Dis. 9, 10-6 (2003).
52. 21-528/S-002, N. Vigamox (moxifloxacin hydrochloride ophthalmic solution) 0.5% as base. 3 (2004).
53. Yang, M., Yang, Y., Lei, M., Ye, C., Zhao, C., Xu, J., Wu, K. & Yu, M. Experimental studies on soft contact lenses for controlled ocular delivery of pirfinedone: in vitro and in vivo. Drug Deliv. 23, 3538-3543 (2016).
54. Chuahan, A. & Kim, J. Contact lenses for extended release of bioactive agents containing diffusion attenuators. 8404265 B2 (2013).
55. Peng, C. C., Kim, J. & Chauhan, A. Extended delivery of hydrophilic drugs from silicone-hydrogel contact lenses containing Vitamin E diffusion barriers. Biomaterials 31, 4032-4047 (2010).
56. Prausnitz, M. R. Permeability of cornea, sciera, and conjunctiva: A literature analysis for drug delivery to the eye. J. Pharm. Sci. 87, 1479-1488 (1998).

1. A contact lens, comprising: a silicone-hydrogel-based lens comprising Vitamin E and at least one medicament, wherein the medicament includes an antibiotic medicament, a steroidal anti-inflammatory medicament, and a non-steroidal anti-inflammatory medicament, or combination thereof, wherein the contact lens is characterized as having a sustained release of the at least one medicament.

2. The contact lens of claim 1, wherein the Vitamin E functions as a sustained release medium for the at least one medicament, wherein the sustained release is for about 2 to 7 days.

3. The contact lens of claim 1, wherein the antibiotic medicament is levofloxacin, wherein the non-steroidal anti-inflammatory medicament is 3etorolac, and steroidal anti-inflammatory medicament is dexamethasone.

4. The contact lens of claim 1, wherein the amount Vitamin E in the contact lens is about 1% to 70%.

5. The contact lens of claim 1, wherein Vitamin E is entrapped in the hydrogel-based lens and wherein the at least one medicament is incorporated into the contact lens.

6. A contact lens, comprising: a hydrogel-based lens comprising Vitamin E and 3etorolac and at least one medicament, wherein the medicament includes an antibiotic medicament, a steroidal anti-inflammatory medicament, and a non-steroidal anti-inflammatory medicament, or combination thereof, wherein contact lens is characterized as having a sustained release of the at least one medicament.

7. The contact lens of claim 6, wherein the Vitamin E functions as a sustained release medium for the at least one medicament, wherein the sustained release is for about 3 to 7 days.

8. The contact lens of claim 6, wherein the antibiotic medicament is levofloxacin and steroidal anti-inflammatory medicament is dexamethasone.

9. The contact lens of claim 6, wherein the amount Vitamin E in the contact lens is about 1 to 70%.

10. The contact lens of claim 6, wherein Vitamin E is entrapped in the hydrogel-based lens and wherein the 4etorolac and at least one medicament is incorporated into the contact lens.

11. A method of preparing a hydrogel-based contact lens, the method comprising:
exposing the hydrogel-based contact lens to a first solution including Vitamin E and a solvent, wherein the solvent swells a hydrogel of the hydrogel-based contact lens so that the Vitamin becomes entrapped by the hydrogel to form a modified contact lens;
exposing the modified contact lens to a second solution including at least one medicament absorbed into the lens matrix to form a final contact lens, wherein the medicament includes an antibiotic medicament, a steroidal anti-inflammatory medicament, and a non-steroidal anti-inflammatory medicament, or combination thereof,
wherein the final contact lens is characterized as having a sustained release of the at least one medicament.

12. The method of claim 11, wherein the exposing the contact lens Vitamin E comprises soaking the contact lens for 1 min to 24 hours at about room temperature with the first solution comprising the Vitamin E and the solvent.

13. The method of claim 11, wherein the exposing the modified contact lens to at least one medicament comprises soaking the contact lens for 1 hour to 10 days at about room temperature with the second solution comprising the at least one medicament.

14. The method of claim 11, wherein the non-steroidal anti-inflammatory medicament is ketorolac, bromofenac, or a combination thereof.

15. The method of claim 11, wherein the antibiotic medicament is levofloxacin and steroidal anti-inflammatory medicament is dexamethasone.

16. The method of claim 11, wherein the antibiotic medicament is moxifloxacin or levoflaxin and steroidal anti-inflammatory medicament is dexamethasone or dexamethasone phosphate and NSAID is ketorolac or bromfenac.

17. The method of claim 11, wherein the amount Vitamin E in the contact lens is about 1% to 70%.

18. The method of claim 11, wherein the silicone-hydrogel is made of one or more of the following: hydroxyethyl methacrylate (HEMA), dimethyl acrylate (DMA), methacrylic acid, or 3-[tris (trimethylsiloxy)silyl]propyl methacrylate (Tris methacrylate).

19. A method of treating an ocular condition, comprising:
positioning a contact lens onto the eye in need of treatment for a period of time, wherein the contact lens is a contact lens as described in claim 1 and
removing the contact lens after the period of time has expired.

20. The method of claim 19, wherein positioning is performed after a procedure on the eye.

21. The method of claim 19, wherein the condition is corneal keratitis or conjunctivitis.
